(19) United States
(12) Patent Application Publication
(10) Pub. No.: US 2005/0232876 A1
(43) Pub. Date: Oct. 20, 2005

(54) Skin care compositions

(76) Inventors: Robin Lynn Minga, Blountville, TN (US); Suzanne Winegar Dobbs, Kingsport, TN (US); Terry Ann Oldfield, Kingsport, TN (US)

Correspondence Address: Jonathan D. Wood;Eastman Chemical Company, P.O. Box 511, Kingsport, TN, 37662-5075, US

(21) Appl. No.: 10/827,691

(22) Filed: Apr. 19, 2004

(51) Int. Cl. A61K 7/42; A61K 7/11; A61K 7/06; A61K 7/50

(52) U.S. Cl. 424/59; 424/70.13

(57) ABSTRACT

Disclosed are skin care compositions comprising an admixture of sucrose acetate isobutyrate and certain diol and glycerol carboxylate esters. The liquid skin care compositions are useful as cosmetic, protective and therapeutic dermatological compositions and comprise an admixture of sucrose acetate isobutyrate and one or more carboxylic acid esters of a polyol selected from propylene glycol and glycerol wherein the carboxylate ester residues contains from 2 to 10 carbon atoms. The skin care compositions exhibit smoothness and water resistance when applied to the skin such as the human body including eyelids and lips.

### FIELD OF THE INVENTION

This invention pertains to skin care compositions comprising an admixture of sucrose acetate isobutyrate and certain diol and glycerol carboxylate esters. More specifically, this invention pertains to liquid skin care compositions such as cosmetic, protective and therapeutic dermatological compositions comprising an admixture of sucrose acetate isobutyrate and one or more carboxylic acid esters of a polyol selected from propylene glycol and glycerol wherein the carboxylate ester residues contains from 2 to 10 carbon atoms. The skin care compositions exhibit smoothness and water resistance when applied to the skin such as the human body including eyelids and lips.

### BACKGROUND OF THE INVENTION

Sucrose acetate isobutyrate (SAIB) is a stable liquid used as a weighting agent in citrus beverages such as fruit juices, energy drinks, and carbonated soft drinks. Weighting agents have a relatively high specific gravity and are mixed with the flavoring oils to stabilize the flavor emulsion and to prevent separation of the flavoring oils from the water in the finished beverage. SAIB (100%) has an extremely high viscosity at room temperature and must be heated to make it pourable before it can be added to the flavor ingredients. U.S. Pat. No. 4,705,691 discloses a beverage opacifier comprising (a) one or more substantially saturated fat as a clouding agent and (b) one or more weighting agent. Component (a) may be glyceryl tri-caprylate/caprate and component (b) may be SAIB. JP-B1-52-35746 discloses a flavor composition for a beverage comprising (1) a flavor oil with (2) a C₈-C₁₄ fatty acid ester of sorbitol, sorbitan or sorbide in an amount of 20-100% to the flavor oil, (3) SAIB and (4) a C₆-C₁₂ saturated fatty acid glyceride in an amount of 5-30% to the SAIB. JP-A1-59-210870 discloses a sports drink composition consisting of 0.1-60 weight percent SAIB and 40-99.9 weight percent of a food composition consisting of 0.01-10 parts by weight Vitamin E and 100 parts by weight of an oil-fat composition consisting of a mixed fat made of 30-95 weight percent of a medium-length triglyceride and 5-70 weight percent of a natural oil-fat or ester exchange oil and more than 80 parts by weight of less than 40 weight percent natural gum aqueous solution.

SAIB (100%) is difficult to incorporate into cosmetic/personal care products because of its high viscosity and because it is extremely tacky. Lipsticks and lip liners containing SAIB are proposed by JP-A1-73-30549. A cosmetic composition comprising an oil, a dehydrated clay material and SAIB is disclosed in JP-A1-61-207314. SAIB must be heated to make it pourable for incorporation into cosmetics and skin care compositions. The high viscosity and, especially, the tackiness (stickiness) of SAIB severely limits its use in skin care products or as a skin care product by itself. WO 99/38489 discloses liquid personal cleaning emulsions containing a weighting oil that may be SAIB. The compositions comprise 1-30% of a lipophilic skin moisturizing agent, 1-20% of a weighting oil, 0.1-5% stabilizer, 1-30% of a lathering agent and water. U.S. Pat. Nos. 5,747,058 and 6,413,536 disclose compositions for the delivery of biologically active substances comprising:
(1) a non-water soluble, high viscosity, liquid carrier material comprising a nonpolymeric ester or mixed ester of one or more carboxylic acids, having a viscosity of at least 5000 cP at 37° C., that does not crystallize neat under ambient or physiological conditions; and
(2) a biologically-active substance.
   Component (1) may be SAIB which may be diluted with an extensive variety of materials such as caprylic/capric triglyceride and propylene glycol dicaprylate/caprate to form a lower viscosity liquid carrier material. Example O of U.S. Pat. No. 6,413,536 discloses a drug delivery composition comprising zidovudine and dideoxycytodine in a 70:30 SAIB/Miglycol® 810 mixture. Miglyol 810 is described elsewhere in the patent as a caprylic/capric triglyceride. If SAIB were easier to handle and had a smoother, less tacky feel on the skin, it could be used more widely in various types of cosmetics/personal care products.

### SUMMARY OF THE INVENTION

We have discovered that the viscosity and tackiness problems that limit the utility of SAIB in skin care compositions can be overcome by blending certain glycerol triesters with SAIB. Such blends have been found to be useful in liquid skin care compositions wherein the blends exhibit smoothness when applied to human skin and excellent resistance to removal with water. These characteristics are advantageous in the formulation of liquid skin care compositions including cosmetics. Thus, in its broader aspects, the present invention provides a liquid skin care composition comprising a pre-blended mixture of SAIB and a glycerol tri-carboxylate ester or propylene glycol di-carboxylate ester wherein the carboxylate residues are residues of carboxylic acids containing 2 to 10 carbon atoms and wherein the composition contains one or more skin care or cosmetic agents selected from colorants, skin emollients, sunscreen agents and therapeutic agents. In another embodiment, the liquid skin care compositions provided by the present invention comprise an oil-in-water emulsion wherein the oil (discontinuous) phase comprises a pre-blended mixture of SAIB and a glycerol tri-carboxylate ester or propylene glycol di-carboxylate ester wherein the carboxylate residues are residues of carboxylic acids containing 2 to 10 carbon atoms and wherein the emulsion contains one or more skin care or cosmetic agents selected from colorants, skin emollients, sunscreen agents and therapeutic agents. We have found that pre-blended mixtures of SAIB and one or more glycerol tri-esters and/or propylene glycol di-esters of carboxylic acids containing 2 to 10 carbon atoms are compatible, i.e., miscible, with SAIB and form clear mixtures that do not separate upon standing for prolonged periods of time. Another embodiment of the present invention is an aerosol skin care composition comprising a pre-blended mixture of SAIB and a glycerol tri-carboxylate ester or a propylene glycol di-carboxylate ester wherein the carboxylate residues are residues of carboxylic acids containing 2 to 10 carbon atoms, one or more skin care or cosmetic agents selected from colorants, skin emollients, sunscreen agents and therapeutic agents, and a propellant.

### DETAILED DESCRIPTION

The relative amounts of SAIB and ester additive, i.e., the glycerol tri-esters and/or propylene glycol di-esters, useful in the present invention are SAIB:ester additive weight ratios of about 80:20 to 70:30. The optimum weight ratio depends on a plurality of factors such as the particular ester additive employed, the end use of the liquid skin care composition and the presence of other ingredients that may contribute to lowering viscosity and/or tackiness. These SAIB blends have a smooth-feel, spread and glide easily, and have a glossy appearance when applied to the skin. The blends described also have a lower viscosity as compared to unmodified SAIB and can be incorporated easily into cosmetic formulations. As a component of a cosmetic composition, the SAIB/ester additive blends provide water resistance and transfer resistance to cosmetic skin care products and color cosmetics. In the case of skin and lip care products, the SAIB/ester additive blends can provide a protective barrier.

Examples of the glycerol tri-esters useful in the present invention include glycerol triacetate (triacetin), glycerol tri-n-butyrate (tributyrin) and glycerol caprylate/caprate, i.e., a glycerol tri-carboxylate wherein the carboxylate residues are mixed caprylate (octanoate) and caprate (decanoate). Examples of the propylene glycol (propane-1,2-diol) di-esters include propylene glycol diacetate, propylene glycol dipropionate and propylene glycol caprylate/caprate, i.e., a propylene glycol dicarboxylate wherein the carboxylate residues are mixed caprylate and caprate. The ester additive preferably is selected from from glycerol tri-carboxylate esters and propylene glycol di-carboxylate esters wherein the carboxylate residues are residues of carboxylic acids selected from acetic acid, butyric acid, isobutyric acid, caprylic acid, caprate acid or a mixture of any two or more threof. The caprylate/caprate mixed esters generally are most preferred. Liquid triglycerides of fatty acids having greater than 10 carbons such as coconut oil, corn oil, and soybean oil, have been found to be incompatible with SAIB. The propylene glycol diester of caprylic and capric acids, commercially available as Captex® 200, Abitec Corp., has been found to provide the most desirable result, effectively reducing the viscosity of SAIB and eliminating its tackiness at a concentration of 20 weight percent in the propylene glycol diester/SAIB blend. The substantivity, transfer resistance, and water resistance of SAIB on the skin are maximized at its maximum concentration. Therefore, the 80/20 weight/weight blend of SAIB/propylene glycol diester is the most preferred embodiment of this invention.

To provide the same degree of smoothness and glide on the skin as the propylene glycol caprylic/capric diester at 20 weight percent typically requires 25 weight percent of the C₂-C₁₀ fatty acid ester triglycerides. At the same concentration, triglycerides of C₄-C₁₀ fatty acid esters provide a smoother feel on the skin, with more glide, than the C₂ fatty acid ester triglyceride (triacetin). Although vegetable oils such as soybean oil, corn oil, and coconut oil are incompatible in blends with SAIB, resulting in haze and/or separation, the glycerol triesters and propylene glycol diesters described above can be added to SAIB/vegetable oil blends to compatibilize and solubilize the SAIB and vegetable oil. For example, a blend of SAIB/glycerol caprylate/caprate/soybean oil at a weight ratio of 80/10/10 is clear and spreads easily on the skin.

The SAIB employed in the present invention is commercially available in both food and industrial grades. The SAIB consists of an average of two acetyl and six isobutyryl residues per sucrose molecule.

The liquid skin care compositions provided by this invention typically comprise about 2 to 95 weight percent of one or more of the preblended SAIB/ester additives described herein. The remainder of the compositions may be selected from various diluents and skin care components so long as the compositions are liquid, e.g., the compositions are in a pourable form. The skin care compositions may be applied directly to the skin as a protectant, as a carrier of other ingredients, and to provide gloss. The SAIB component of the liquid skin care compositions can function as a skin protectant in products such as skin moisturizers, baby skin care products, body oils, and after-bath and shower products. The SAIB component also can carry and hold other ingredients on the skin, such as ultraviolet light absorbers in the case of sunscreen products or dihydroxyacetone in sun-less tanning products. Also, the SAIB/ester additive mixture may be incorporated into face makeup such as liquid foundation, blushers, eye shadow, eyeliner, or mascara; and in body makeup. In these products, the SAIB/diluent blend provides gloss, transfer resistance, and water resistance. The liquid skin care compositions of the present invention also include baby care preparations, bath and shower products and body oils. The liquid skin care compositions preferably comprise about 2 to 95 weight percent of one or more of the preblended SAIB/ester additives, about 0.5 to 20 weight percent of one or more skin care or cosmetic agents selected from colorants, skin emollients, sunscreen agents and therapeutic agents, and about 5 to 98 weight percent of one or more dermatologically acceptable liquid diluents. Examples of such dermatologically acceptable liquid diluents include homogeneous mixtures or emulsions including water, fatty acids, fatty acid esters, vegetable oil, fatty alcohols, ethoxylated and/or propoxylated fatty alcohols, glycol ethers, glycol esters, glyceryl esters, liquid hydrocarbons such as mineral oil, squalane, and squalene, silicone oils, alcohol solvents such as ethanol and isopropanol, and/or liquid propellants.

In addition to the ingredients discussed above, the liquid skin care compositions may contain other ingredients commonly present in products intended for application to human skin. Examples of such other optional ingredients include fragrances, emulsifiers, surfactants, humectants, skin conditioning agents, skin protectants, skin-lightening agents, polymeric film formers, film modifiers such as plasticizers, viscosity increasing or decreasing agents, binders, waxes, solvents, colorants, preservatives, and vitamins.

The blends of SAIB and the glycerol tri-carboxylate ester or propylene glycol di-carboxylate ester may be prepared by first heating the SAIB to about 60° C. to make it pourable. The SAIB and ester additive are weighed into a jar and mixed together. The blend then is heated and mixed to ensure homogeneity.

The oil-in-water emulsion of the present invention comprises a discontinuous oil phase and a continuous aqueous phase wherein the SAIB/ester additive constitutes about 2 to 25 weight percent, based on the total weight of the emulsion. The oil or organic phase comprises a pre-blended mixture of SAIB and a glycerol tri-carboxylate ester or propylene glycol di-carboxylate ester wherein the carboxylate residues are residues of carboxylic acids containing 2 to 10 carbon atoms. The oil phase typically comprises one or more oils or oil-soluble materials that are compatible with the SAIB/ester additive blend and form a stable emulsion. Examples of such oils and oil-soluble compounds include alkyl esters of fatty acids containing a total of about 12 to 38 carbon atoms, essential oils, fatty acids, vegetable oils, animal and fish oils, fatty alcohols, ethoxylated and/or propoxylated fatty alcohols, glyceryl mono- and di-esters, liquid hydrocarbons such as mineral oil, squalane and squalene, lanolin and its derivatives, silicone oils and the like. In some cases, the combination of oils and/or oil-soluble materials are compatible with the SAIB/ester additive although individual components may not be. Specific examples of such liquid lipophillic materials are listed in the International Cosmetic Dictionary and Handbook of the Cosmetic, Toiletry, and Fragrance Association (CTFA) and include octyl palmitate, isopropyl palmitate, isopropyl myristate and stearyl stearate. The emulsion contains one or more skin care or cosmetic agents selected from colorants, skin emollients, sunscreen agents and therapeutic agents, either in the oil phase or water phase, depending upon the solubility of the agent.

The emulsions provided by our invention are comprised of about 50 to 95 weight percent of the aqueous phase and about 5 to 50 weight percent of the oil phase (including additives). Preferably, the aqueous phase comprises about 60 to 90 weight percent and the oil phase comprises about 10 to 40 weight percent of the emulsion. The emulsions may be prepared by conventional procedures well-known to those skilled in the art. Various surfactants may be utilized as the emulsifying agent(s) in the emulsions.

Another embodiment of the present invention pertains to a liquid composition designed for application to the skin or hair in the form of a spray, i.e., in a finally divided form. Such sprayable compositions may be applied to the skin or hair by means of either a pump spray or by means of an aerosol. The sprayable compositions comprise a pre-blended mixture of SAIB and ester additive dissolved in a solvent and/or propellant. The sprayable liquid compositions also contain one or more skin care or cosmetic agents selected from colorants, skin emollients, sunscreen agents and therapeutic agents. The SAIB/ester additive mixture may be sprayed onto the skin or hair to provide gloss and/or to carry and fix on the skin or hair an active ingredient or decorative pigment. Such sprayable compositions may contain one or more solvents and/or propellants. Examples of solvents include ethanol, isopropanol, mixtures of ethanol and water, mixtures of isopropanol and water, C₁-C₆ alkyl acetates, glycol ethers such as diethylene glycol monoethyl ether, 2-butoxyethanol, and 1-methyoxy-2-isopropanol, and C₃-C₈ ketones such as acetone. The preferred solvent is ethanol or a mixture of ethanol and water. The preferred mixture of ethanol and water should have a ratio of ethanol to water high enough to maintain solubility of the SAIB/ester additive mixture. For example, if the SAIB/ester additive concentration is about 6 weight percent, the weight ratio of ethanol to water should be at least about 4:1. Examples of propellants include propane, n-butane, isobutane, hexane, carbon dioxide, nitrous oxide, dimethyl ether, diethyl ether and fluorochlorohydrocarbons. The preferred propellant is dimethyl ether. Active ingredients include ingredients such as UV absorbers, dihydroxyacetone, skin bleaching agents, antiacne agents, biocides, and pesticides. Decorative pigments include colorants such as inorganic pigments, synthetic organic colorants, and the lakes of organic colorants; and special effect pigments such as mica, coated mica, metal flakes and powders, and bismuth oxychloride that provide effects such as sparkle, pearlescence, or fluorescence.

The body or hair spray composition typically comprises about 5 to 65 weight percent of the pre-blended mixture of SAIB and ester additive, based on the total weight of the spray composition. Other ingredients or agents can comprise up to 60, e.g., from about 5 to 60, weight percent of the spray composition. The remainder of the spray composition consists of the solvent or propellant or mixture of solvents and/or propellants.

### EXAMPLES

The preparation and use of the liquid skin care compositions of the present invention is further illustrate by the following examples wherein all percentages are by weight unless specified otherwise.

### Example 1

Various blends of SAIB and glycerol and propylene glycol esters were prepared as described above by first heating the SAIB to about 60° C. to make it pourable. The SAIB and ester additive were weighed into a jar and mixed together. The mixture then was heated and mixed to ensure homogeneity. The blends prepared are shown in Table I. For those blends in Table I indicated as being cloudy or separated, the cloudiness or separation developed as, or after, the mixture cooled to room temperature. Those blends indicated as providing a smooth feel on the skin had a glossy appearance, and provided a water-resistant coating on the skin that was difficult to wash off with soap and water. The water-resistance of the coating on the skin was apparent from the appearance of water beads as water was rinsed over the skin. The same effects of smoothness, gliding, gloss, and water resistance were noted when these mixtures were applied to the lips.

TABLE I

| | Weight Ratio of Ester Additive:SAIB | | |
|---|---|---|---|
| Ester Additive | 20:80 | 25:75 | 30:70 |
| Glycerol Triacetate | Spreads, | Smooth | Smooth |
| | slightly tacky | | |
| Glycerol Tributyrate | Smooth | Smooth | Smooth |
| Glycerol Tricaprylate/caprate¹ | Spread, | Smooth | Smooth |
| | slightly tacky | | |
| Glycerol Tricaprylate/caprate² | Spreads, | Smooth | Smooth |
| | slightly tacky | | |
| Coconut Oil | Hazy, tacky | — | — |
| Soybean Oil | Separated | Separated | Separated |
| Corn Oil | Separated | Separated | Separated |
| Glycerol Mono/Di-C18 Ester³ | Hazy, tacky | — | — |
| Propylene Glycol | Smooth | Smooth | Smooth |
| Dicaprylate/caprate⁴ | | | |
| Acetyl Tributyl Citrate⁵ | Tacky | — | — |
| Triethylene Glycol | Separated | — | — |
| Mixture of 1 part | Hazy, | — | — |
| Glycerol Tricaprylate/ | separated | | |
| caprate & | | | |
| 4 parts Soybean Oil⁶ | | | |
| Mixture of 1 part | Clear, | — | — |
| Glycerol Tricaprylate/ | slightly tacky | | |
| caprate & | | | |
| 1 part Soybean Oil | | | |
| Mixture of 1 part | Hazy | — | — |
| Glycerol Triacetate & | | | |
| 4 parts Soybean Oil | | | |
| Mixture of 1 part | Hazy, thick | — | — |
| Glycerol Triacetate & | | | |
| 1 part Soybean Oil | | | |
| Mixture of 4 parts | Hazy | — | — |
| Glycerol Triacetate & | | | |
| 1 part Soybean Oil | | | |
| Mixture of 10 parts | Clear, smooth | — | — |
| Glycerol Triacetate & | | | |
| 1 part Soybean Oil | | | |

¹Captex 300
²Neobee M-5
³Drewmulse GMO-K
⁴Captex 200
⁵Citroflex A-4
⁶Mixtures are parts by weight

### Example 2

The contact angle of water on films of the SAIB/ester additive blends was measured to show the water resistant nature of these mixtures. Contact angle is a measure of the surface wettability and is described in Test Method ASTM D5725-99. For each blend listed in Table II, 0.2 g of SAIB/ester additive blend was weighed onto a piece of 50 pound Kraft paper measuring 28.3 cm². The blend was spread with the finger to cover the surface of the Kraft paper. Water droplets were dropped onto the coated Kraft paper. The contact angle of each drop resting on the surface of the coated paper was measured on an enlarged video image of each drop displayed on a monitor. Water droplets dropped onto uncoated Kraft paper quickly soaked into the paper. Therefore, the contact angle of water on uncoated paper is effectively 0°. Contact angles for coated paper are given in Table 2. A higher contact angle indicates less wettability and greater water resistance of the SAIB/ester additive blend. A trend of reduced contact angle with increased level of glycerol triacetate and tributyrate is observed. This trend is not apparent with glycerol tricaprylate/caprate and propylene glycol di-caprylate/caprate. This can be attributed to the more hydrophilic nature of glycerol triacetate and tributyrate compared to the other two ester additives.

TABLE II

| | Weight Ratio of Ester Additive:SAIB | | | |
|---|---|---|---|---|
| Ester Additive | 15:85 | 20:80 | 25:75 | 30:70 |
| Glycerol Triacetate | 68° | 65° | 62° | 58° |
| Glycerol Tributyrate | — | 68° | 63° | 62° |
| Glycerol Tricaprylate/ | 65° | 67° | 63° | — |
| caprate | | | | |
| Propylene Glycol | 66° | — | 70° | 66° |
| Tricaprylate/caprate | | | | |

### Example 3

Water vapor transmission rates (WVTR) were measured on films of the SAIB/ester additive blends to determine if the blends improve moisture retention in the skin. The WVTR of Vaseline® Intensive Care® Water Resistant Skin Protectant Lotion (VIC) was measured for comparison. For each blend and the VIC lotion evaluated, 0.5 g of the blend of the VIC lotion was weighed into the center of a piece of Kraft paper, 9.5 cm in diameter. The mixture was spread with the finger to cover the surface of the Kraft paper. WVTR was determined according to ASTM E96. Conditions of the test were 32° C. and 50% room humidity. The results are given in Table III wherein the values reported for WVTR are g/m²/24 hours.

TABLE III

| Ester Additive/SAIB Blend | WVTR |
|---|---|
| 20/80 Glycerol Triacetate/SAIB | 601 |
| 20/80 Glycerol Tricaprylate/caprate/SAIB | 483, 570* |
| VIC Lotion | 759 |
| Kraft Paper, Uncoated | 1749 |

*Two determinations at different times

### Example 4

In a glass aerosol container, 36.1-g 80/20 SAIB/glycerol tricaprylate/caprate blend was mixed with 2.9-g 2-ethylhexyl p-methoxycinnamate. The combined 2-ethylhexyl p-methoxycinnamate and SAIB/glycerol ester was very viscous. Dimethyl ether propellant (21.0 g or 31.8 ml) was added and mixed with the other ingredients, resulting in a clear solution having a much-reduced viscosity. The mixture was sprayed onto the skin. The spray-out was a fine mist. The material left on the skin was slightly tacky, but spread easily for good coverage.

### Example 5

A sunscreen emulsion was prepared from the following ingredients:

| | | | |
|---|---|---|---|
| Ingredient | % | Grams | Trade Name |

PART 1

| | | | |
|---|---|---|---|
| Millipore Water | 64.9 | 324.5 | |
| Propylene Glycol | 2.0 | 10.0 | Propylene Glycol |
| Carbomer | 0.2 | 1.0 | Carbopol Ultrez 10 |
| Propylparaben | 0.1 | 0.5 | Propylparaben |
| Methylparaben | 0.1 | 0.5 | Methylparaben |
| Glyceryl Dilaurate | 0.5 | 2.5 | Lexemul GDL |

PART 2

| | | | |
|---|---|---|---|
| Octyl Palmitate | 8.0 | 40.0 | Ceraphyl 368 |
| Ethylhexyl Methoxycinnamate | 7.5 | 37.5 | Parsol MCX |
| Benzophenone-3 | 3.0 | 15.0 | Escalol 567 |
| 80/20 SAIB/Glycerol | 5.0 | 25.0 | |
| Tricaprylate/caprate | | | |
| Cetearyl Alcohol (and) | 1.5 | 7.5 | Promulgen D |
| Ceteareth-20 | | | |
| Glyceryl Dilaurate | 1.5 | 7.5 | Lexemul GDL |
| Stearic Acid | 1.0 | 5.0 | Palmac 90-18 |
| Glyceryl Stearate & Laureth-23 | 2.0 | 10.0 | Cerasynt 945 |
| Dimethicone | 1.0 | 5.0 | D.C. 193 surfactant |

PART 3

| | | | |
|---|---|---|---|
| Triethanolamine (50% in water) | 1.2 | 6.0 | Triethanolamine |

PART 4

| | | | |
|---|---|---|---|
| Propylene Glycol (and) | 0.5 | 2.5 | Germaben II |
| Diazolidinyl Urea (and) | | | |
| Methylparaben (and) | | | |
| Propylparaben | | | |
| Total | 100.0 | 500.0 | |

All of the ingredients of Part 1 except the Carbopol Ultrez 10 were combined in a beaker at ambient temperature while stirring with a propeller stirrer blade. The Carbomer was slowly sifted in with continued stirring and the mixture was heated to approximately 75° C. The ingredients of Part 2 were combined in another beaker and heated to approximately 75° C. with stirring to melt all ingredients. The mixture of the Part 2 ingredients was added quickly to the Part 1 ingredients while stirring. Stirring was continued while the mixture cooled. When the temperature of the mixture reached approximately 50° C., the aqueous triethanolamine of Part 3 was added. The propeller stirrer blade was changed to a zig-zag blade as viscosity increased. The ingredients of Part 4 were added and mixing was continued to achieve complete mixing. The resulting emulsion was cooled in an ice bath with continued mixing for 30 minutes. This sunscreen emulsion is a white, medium viscosity lotion having a pH of 7.4. When rubbed on the skin, it spreads easily, is readily absorbed by the skin, and imparts a pleasant feel. The applied emulsion does not feel tacky.

### Example 6

A moisturizing cream emulsion was prepared from the following ingredients:

| | | | |
|---|---|---|---|
| Ingredient | % | Grams | Trade Name |

PART 1

| | | | |
|---|---|---|---|
| Millipore Water | 83.35 | 500.1 | |
| Glycerin | 2.00 | 12.0 | Glycerol |
| PEG-75 Lanolin | 0.50 | 3.0 | Solulan 75 |
| Carbomer | 0.20 | 1.2 | Carbopol Ultrez 10 |

PART 2

| | | | |
|---|---|---|---|
| Cetearyl Alcohol (and) | 2.00 | 12.0 | Promulgen D |
| Ceteareth-20 | | | |
| Glyceryl Dilaurate | 0.50 | 3.0 | Lexemul GDL |
| Cetyl Alcohol | 1.50 | 9.0 | Crodacol C-95-NF |
| Stearic Acid | 1.00 | 6.0 | Palmac 90-18 |
| | | | D.C. 200 Fluid 350 |
| Dimethicone | 0.20 | 1.2 | cSt. |

PART 3

| | | | |
|---|---|---|---|
| Sodium Hydroxide, 50% | 0.25 | 1.5 | Sodium Hydroxide |

PART 4

| | | | |
|---|---|---|---|
| Coco-Caprylate/Caprate¹ | 3.00 | 18.0 | Cetiol LC |
| 80/20 SAIB/Glycerol | 5.00 | 30.0 | |
| Tricaprylate/caprate | | | |

PART 5

| | | | |
|---|---|---|---|
| Propylene Glycol (and) | 0.50 | 3.0 | Germaben II |
| Diazolidinyl Urea (and) | | | |
| Methylparaben (and) | | | |
| Propylparaben | | | |
| | 100.00 | 600.0 | |

¹Mixed caprylate/caprate esters of coconut alcohol

All of the ingredients of Part 1 except the Carbopol Ultrez 10 were combined in a beaker at ambient temperature while stirring with a propeller stirrer blade. The Carbopol was slowly sifted into the Part 1 mixture with continued stirring and the mixture was heated to approximately 75° C. The ingredients of Part 2 were combined in another beaker and heated to approximately 80° C. while stirring to melt all ingredients. The Part 2 mixture was added quickly to the Part 1 mixture while stirring. The resulting mixture of Parts 1 and 2 was allowed to cool to approximately 70° C. while stirring was continued. When the combined Parts 1 and 2 reached 70° C., Part 3 was added with stirring. When the temperature of the resulting mixture (Parts 1, 2, and 3) had decreased to approximately 55° C., the Part 4 ingredients (combined and heated to ˜55° C.) were added. The propeller stirrer blade was changed to a zig-zag blade as viscosity increased. Part 5 was added and stirring was continued to achieve complete mixing. The final moisturizing cream emulsion was cooled in an ice water bath with continued mixing for 30 minutes. This moisturizing cream is a white, medium viscosity cream. When rubbed on the skin, it spreads easily, is readily absorbed by the skin, and imparts a pleasant feel. The applied cream does not feel tacky.

The invention has been described in detail with particular reference to preferred embodiments thereof, but it will be understood that variations and modifications can be effected within the spirit and scope of the invention.

1. A liquid skin care composition comprising a pre-blended mixture of sucrose acetate isobutyrate (SAIB) and an ester additive selected from glycerol tri-carboxylate esters or propylene glycol di-carboxylate esters wherein the carboxylate residues are residues of carboxylic acids containing 2 to 10 carbon atoms and wherein the composition contains one or more skin care or cosmetic agents selected from colorants, skin emollients, sunscreen agents and therapeutic agents.

2. The liquid skin care composition according to claim 1 wherein the pre-blended mixture of SAIB and the ester additive constitutes about 2 to 95 weight percent of the composition and the carboxylate residues are residues of carboxylic acids selected from acetic acid, butyric acid, isobutyric acid, caprylic acid, caprate acid or a mixture of any two or more threof.

3. The liquid skin care composition according to claim 2 wherein the weight ratio of SAIB:ester additive is in the range of about 80:20 to 70:30.

4. A liquid skin care composition comprising an oil-in-water emulsion wherein the oil phase comprises a pre-blended mixture of SAIB and a glycerol tri-carboxylate ester or propylene glycol di-carboxylate ester wherein the carboxylate residues are residues of carboxylic acids containing 2 to 10 carbon atoms and wherein the emulsion contains one or more skin care or cosmetic agents selected from colorants, skin emollients, sunscreen agents and therapeutic agents.

5. The liquid skin care composition according to claim 1 wherein the composition is an emulsion and the pre-blended mixture of SAIB and glycerol tri-carboxylate ester or propylene glycol di-carboxylate ester constitutes about 2 to 25 weight percent of the composition and the carboxylate residues are residues of carboxylic acids selected from acetic acid, butyric acid, isobutyric acid, caprylic acid, caprate acid or a mixture of any two or more thereof.

6. An oil-in-water skin care emulsion comprising about 50 to 95 weight percent aqueous phase and about 5 to 50 weight percent oil phase containing in the oil phase a pre-blended mixture of SAIB and an ester additive selected from glycerol tri-carboxylate ester or propylene glycol di-carboxylate ester wherein the carboxylate residues are residues of carboxylic acids containing 2 to 10 carbon atoms and wherein the emulsion contains one or more skin care or cosmetic agents selected from colorants, skin emollients, sunscreen agents and therapeutic agents.

7. An emulsion according to claim 6 wherein the pre-blended mixture of SAIB and the ester additive constitutes about 2 to 95 weight percent of the emulsion and the carboxylate residues are residues of carboxylic acids selected from acetic acid, butyric acid, isobutyric acid, caprylic acid, caprate acid or a mixture of any two or more thereof.

8. An emulsion according to claim 7 comprising about 50 to 95 weight percent aqueous phase and about 5 to 50 weight percent oil phase.

9. A sprayable liquid skin or hair care composition comprising a solution of a pre-blended mixture of sucrose acetate isobutyrate (SAIB) and an ester additive selected from glycerol tri-carboxylate ester or propylene glycol di-carboxylate ester in a solvent, a propellant or a mixture thereof, wherein the carboxylate residues are residues of carboxylic acids containing 2 to 10 carbon atoms and wherein the solution contains one or more skin care or cosmetic agents selected from colorants, skin emollients, sunscreen agents and therapeutic agents.

10. A sprayable composition according to claim 9 wherein the composition is sprayable by means of a pump and comprises about 5 to 65 weight percent of the pre-blended mixture of SAIB and an ester additive dissolved in ethanol, water/ethanol mixtures, isopropanol or isopropanol/water mixtures wherein the weight ratio of SAIB:ester additive is in the range of about 80:20 to 70:30.

11. A sprayable composition according to claim 9 wherein the composition is sprayable by means of an aerosol propellant and comprises about 5 to 65 weight percent of the pre-blended mixture of SAIB and an ester additive dissolved in a propellant selected from propane, n-butane, isobutane, hexane, carbon dioxide, nitrous oxide, dimethyl ether, diethyl ether and fluorochlorohydrocarbons wherein the weight ratio of SAIB:ester additive is in the range of about 80:20 to 70:30.
